SEQUENCE LISTING (6 sequences)

SEQ ID NO: 1 - protein, 11 aa, Artificial
  First peptide fragment
GQPAGPDNKEN

SEQ ID NO: 2 - protein, 10 aa, Artificial
  Second peptide fragment
GEAVAARKLF

SEQ ID NO: 3 - protein, 12 aa, Artificial
  Third peptide fragment
NCPVPGKPGEAV

SEQ ID NO: 4 - protein, 10 aa, Artificial
  NCPVPGKPGE
NCPVPGKPGE

SEQ ID NO: 5 - protein, 10 aa, Artificial
  Fifth peptide fragment
PVPGKPGEAV

SEQ ID NO: 6 - protein, 31 aa, Artificial
  Antigen
GQPAGPDNKENCPVPGKPGEAVAARKLFAPQ

(19) United States
(12) Patent Application Publication
(10) Pub. No.: US 2021/0190780 A1
(43) Pub. Date: Jun. 24, 2021

(54) SERUM THYMIDINE KINASE 1 DETECTION KIT BASED ON AUTOMATIC CHEMILUMINESCENCE ANALYZER

(71) Applicant: SINO-SWED TONGKANG BIO-TECH (SHENZHEN) LIMITED, Shenzhen (CN)

(72) Inventors: Li DANG, Shenzhen (CN); Liwen LIANG, Shenzhen (CN); Zuosheng LI, Shenzhen (CN); Huijun LI, Shenzhen (CN); Hu CHEN, Shenzhen (CN); Cong FANG, Shenzhen (CN); Lu CHEN, Shenzhen (CN); Ellen HE, Shenzhen (CN); Sven Isac SKOG, Shenzhen (CN); Ji ZHOU, Shenzhen (CN)

(21) Appl. No.: 17/190,433

(22) Filed: Mar. 3, 2021

Related U.S. Application Data
(63) Continuation of application No. PCT/CN2019/102618, filed on Aug. 26, 2019.

(30) Foreign Application Priority Data
Sep. 4, 2018 (CN) 201811026799.6

(51) Int. Cl. G01N 33/563; C07K 16/40; C07K 16/46; G01N 33/543; G01N 33/573

(52) U.S. Cl. CPC G01N 33/563; C07K 16/40; C07K 16/468; G01N 2333/9122; G01N 33/573; C12Y 207/01021; G01N 33/54326

(57) ABSTRACT

Disclosed are a kit and use thereof. The kit includes a first polyclonal antibody that has been immobilized or suitable for immobilization on a solid carrier, a second polyclonal antibody labeled with a marker. Both the first polyclonal antibody and the second polyclonal antibody are both chicken anti human-thymidine kinase IgY-polyclonal antibodies. Both the first polyclonal antibody and the second polyclonal antibody are suitable for specifically binding to thymidine kinase 1. The kit is suitable for the risk assessment of micro malignant tumors/precancerous diseases and tumors that are not detectable by human population screening images.

### CROSS-REFERENCE TO RELATED APPLICATIONS

This application is a Continuation Application of International Application No. PCT/CN2019/102618, filed on Aug. 26, 2019, which claims priority to Chinese Application No. 201811026799.6, filed on Sep. 4, 2018, the entire disclosure of which is incorporated herein by reference.

This application contains a sequence listing submitted as an ASCII text file, named “Seq-List.txt” and created on Mar. 3, 2021, with 2 kilobytes in size. The material in the above-identified ASCII text file is incorporated herein by reference in its entirety.

### TECHNICAL FIELD

The present disclosure relates to the field of biological detection, and relates to the development of a kit of thymidine kinase 1 in serum for an automatic chemiluminescence analyzer of magnetic particle immune sandwich method technology and the effectiveness of its application in routine human group examination and screening.

### BACKGROUND

Immune magnetic particle separation technology is an immunological technology based on antigen-antibody specific binding. It mainly relies on the modified groups labeled on the surface of magnetic particles, such as amino, carboxyl, tosyl, streptavidin and other groups to be covalently or non-covalently coupled to the labeled antibody, which can be used to bind specific antigens and separate corresponding antigen substances in the automatic chemiluminescence analyzer.

Thymidine kinase 1 is a kinase that is expressed in the cytoplasm and converts deoxythymidine (dThd) into deoxy-thymidylate phosphate (dTMP), which is the only key kinase that dThd is introduced into DNA synthesis through the salvage pathway. The expression of TK1 is closely related to the cell cycle, and its regulation during the cell cycle ensures the supply of 3-phosphothymidylate in DNA replication. This approach supplements the need for DNA synthesis when tumor cells proliferate abnormally. The level of TK1 is closely related to the speed of DNA synthesis in the S phase of the cell cycle. The level of TK1 in tumor cells in the proliferation phase begins to increase at the junction of the G₁ and S phases of the cell cycle. As the cell enters the late G₁ phase, the level of TK1 enzyme gradually rises sharply until the junction of the S and G₂ phases. Therefore, TK1 enzyme is called S-phase special enzyme. Detecting the content of thymidine kinase 1 in the serum can effectively screen out the high-risk groups of precancerous diseases that will progress to malignant tumors in the health checkup in the future, which can also be used in clinical departments to monitor the curative effect, prognosis and risk of recurrence of patients with malignant tumors. However, the existing detection system for detecting thymidine kinase 1 cannot be automated and has low repeat accuracy, which affects the judgment of the detection result. Therefore, the kit for detecting thymidine kinase 1 needs to be improved.

### SUMMARY

The objective of the present disclosure is to provide a kit that can be applied to an automatic chemiluminescence analyzer, has high detection accuracy, simple and convenient operation, and is suitable for routine large-scale population screening.

Cancer diseases are characterized by abnormal cell proliferation. Multiple gene mutations related to cell growth regulation pathways in certain enzymes and proteins lead to uncontrolled regulation of normal cells, leading to the development of abnormal proliferation of malignant tumors. The research and development of new tumor proliferation markers and the establishment of detection technologies are one of the important topics in tumor precision detection and preventive medicine. As early as 1950, thymidine kinase 1 (TK1) was discovered and recognized as a precise protein molecular target for evaluating the rate of cell proliferation. The enzyme of the TK1-pyrimidine salvage pathway catalyzes the conversion of thymidine (dThd) to thymidine monophosphate (dTMP). TK1 is known as a key enzyme for DNA synthesis and a specific enzyme for S phase in the mammalian cell cycle. The level of TK1 is closely related to the rate of DNA synthesis, and then closely related to the proliferation of tumor cells. The inventor designed the human TK1 antigen, successfully prepared the chicken anti-human TK1 IgY polyclonal-antibody, and established a commercial highly sensitive immune enhanced chemiluminescence dot blot (ECL dot blot) detection kit to determine the concentration of TK1 in the serum. It is proved that this detection system is suitable for monitoring the efficacy of various tumors, the recurrence and survival rate of cancer patients. It is especially suitable for early detection of malignant tumors of precancerous diseases and predicting the risk process of tumor development, and is a preferred biomarker for predicting the risk of tumor dynamic process. Based on a meta-study of 35,365 people with big data serum samples from the physical examination population, the inventors set STK1p (serum thymidine kinase 1 protein)=2 pmol/L as a reasonable “risk threshold”, which effectively evaluates the growth rate of abnormal cells in routine population screening. The incidence of new malignant tumors occurring within 6 years is 3-5 times higher than the new incidence rate of Chinese tumor statistics (0.2%-0.3%). Compared with the STK1p low-risk group (≤2 pmol/L), the STK1p high-risk group has an even higher risk (44 times) of developing new malignancies within 11 years. The published meta-study of 16,086 physical examinations has further verified that this test kit is suitable for early tumor detection, including precancerous diseases, tumor-related risk diseases and early microscopic latent malignant tumors. But this method is still semi-automated, especially the 3 microliters of serum spotting requires meticulous manual technical operation, the repeatability is not ideal (the standard error of detection sometimes exceeds 15%), and it is not suitable for routine large-scale population screening.

Therefore, according to an aspect of the present disclosure, the present disclosure provides a kit. According to an embodiment of the present disclosure, the kit includes: a first polyclonal antibody that has been immobilized or is suitable for immobilization on a solid carrier; and a second polyclonal antibody labeled with a marker, both the first polyclonal antibody and the second polyclonal antibody are chicken anti-human thymidine kinase 1 IgY-polyclonal antibody, and both the first polyclonal antibody and the second polyclonal antibody are suitable for specifically binding to thymidine kinase 1.

According to the kit of the embodiment of the present disclosure, by screening a large number of thymidine kinase 1 (TK1) antibodies, the inventor found that the first polyclonal antibody and the second polyclonal antibody, the two chicken anti-human TK1 IgY-polyclonal antibodies, can simultaneously recognize different microregions of the epitope of TK1. That is, two polyclonal antibodies from the same source can simultaneously specifically bind to different surface determinants of the antigen, thereby forming a double antibody sandwich complex. The concentration of the TK1 antigen in the sample is determined based on the light intensity of the luminescence marker attached to the polyclonal antibody labeled with the luminescence marker in the detection complex. The inventor found that chicken anti-human thymidine kinase 1 IgY-polyclonal antibody has better sensitivity and specificity for binding to TK1 in serum than TK1 IgG-monoclonal antibody derived from other animals.

Further, the kit of the embodiment of the present disclosure has high detection sensitivity, strong specificity, short detection time, low detection cost, automatic operation and easy promotion. According to the embodiments of the present disclosure, the detection results of the kits based on the embodiments of the present disclosure can be effectively used for screening of human populations, distinguishing patients with tumor disease risk progression and non-tumor diseases, and the detection repeat accuracy rate is high.

Further, the kit of the embodiment of the present disclosure is used in conjunction with an automatic chemiluminescence immunoassay analyzer to detect thymidine kinase 1 in the sample, thereby realizing full automation of the detection process and reducing errors caused by human operation.

It should be noted that the present disclosure uses two polyclonal antibodies, the first polyclonal antibody and the second polyclonal antibody, to form a diabody sandwich complex, instead of the existing mouse-derived monoclonal antibody and polyclonal antibody to form a diabody sandwich complex. More and more cancer patients are now using monoclonal mouse antibodies for immunotherapy. One of the interfering factors is human anti-mouse IgG monoclonal antibody (HAMA), which often causes antibody response effects. In terms of tumor treatment, especially when mouse monoclonal antibodies are used for treatment, the use of monoclonal antibody-type tumor-related markers for detection will increase the incidence of HAMA in the body. The advantage of the chicken antibody detection method to monitor the efficacy is that it does not react with HAMA. Therefore, the immunoassay method using chicken antibodies in the kit of the embodiment of the present disclosure should theoretically be better than using mammalian antibodies. The chicken anti-human thymidine kinase 1 IgY-polyclonal antibodies shows more advantages than single antibody detection, and the detection results are more accurate.

It should be noted that the first and second polyclonal antibodies in the embodiments of the present disclosure are chicken anti-human thymidine kinase 1 IgY-polyclonal antibodies (hTK1-IgY pAb), and using the antibodies has at least one of the following advantages:

(1) There are molecular genetic differences between chicken IgY and human IgG;

(2) There are species differences between chicken TK1 and human TK1;

(3) Compared with polyclonal antibodies prepared by traditional rabbit immunization, IgY has endogenous molecular homogeneity (only one type of antibody molecule is produced, namely IgY);

(4) IgY antibody does not activate the human complement system, thereby partially blocking the activation of non-specific antigen binding sites in human serum;

(5) Rheumatoid factor (RF) does not react with IgY antibodies. This RF is the main source of non-specific reactions in many immunoassays, since RF partially reacts with the Fc of mammalian antibody IgG, which can lead to false positives in the serum of patients and healthy people.

Besides, the kit according to the above embodiment of the present disclosure may also have the following additional technical features:

According to an embodiment of the present disclosure, epitopes recognized by the first polyclonal antibody and the second polyclonal antibody include a third peptide fragment at a carbon terminal. The third peptide fragment includes a sequence SEQ ID NO: 3, which is a very key specific epitope in the form of serum TK1. The sequence is as follows: NCPVPGKPGEAV (SEQ ID NO: 3)

The epitopes further include at least two peptide fragments selected from the following peptide fragments of thymidine kinase 1: a first peptide fragment at the carbon terminal, a second peptide fragment at the carbon terminal, a fourth peptide fragment at the carbon terminal, and a fifth peptide fragment at the carbon terminal. The first peptide fragment is an accessible epitope, and includes a sequence SEQ ID NO: 1 shown as follows: GQPAGPDNKEN (SEQ ID NO: 1). The second peptide fragment includes a sequence SEQ ID NO: 2 shown as follows: GEAVAARKLF (SEQ ID NO: 2). The fourth peptide fragment includes a sequence SEQ ID NO: 4 shown as follows: NCPVPGKPGE (SEQ ID NO: 4). The fifth peptide fragment includes a sequence SEQ ID NO: 5 shown as follows: PVPGKPGEAV (SEQ ID NO: 5).

According to an embodiment of the present disclosure, the first polyclonal antibody and the second polyclonal antibody are obtained by immunizing chickens with a first antigen. The antigen is a polypeptide at the carbon terminal of human thymidine kinase 1. The inventor screened a large number of different peptides of thymidine kinase 1 and found that the antibody obtained by immunization with the peptide of thymidine kinase 1 located at the C-terminus has good specificity and sensitivity for binding to hTK1. The antibody can bind to hTK1 in the serum, thereby realizing the detection of hTK1 in the serum. The antibodies obtained by immunization with other peptides are almost difficult to bind to serum hTK1, and it is impossible to effectively detect hTK1 in serum. After 15 years of clinical practice and analysis of more than 100,000 samples, the antibody concluded that the antibody can effectively recognize serum hTK1 and distinguish people at high risk of proliferation.

According to an embodiment of the present disclosure, the antigen includes a sequence SEQ ID NO: 6 shown as follows: GQPAGPDNKENCPVPGKPGEAVAARKLFAPQ (SEQ ID NO: 6).

Further, the epitope that the antigen can recognize contains 4 specific epitopes and one accessible epitope. The four specific epitopes and one accessible epitope are: the first peptide fragment at the carbon terminal, one accessible epitope, the second peptide fragment at the carbon terminal, the third peptide fragment at the carbon terminal, the fourth peptide fragment at the carbon terminal, and the fifth peptide fragment at the carbon terminal.

The first peptide fragment includes the sequence SEQ ID NO: 1 shown as follows: GQPAGPDNKEN (SEQ ID NO: 1).

The second peptide fragment includes the sequence SEQ ID NO: 2 shown as follows: GEAVAARKLF (SEQ ID NO: 2).

The third peptide fragment includes the sequence SEQ ID NO: 3 shown as follows: NCPVPGKPGEAV (SEQ ID NO: 3), which is a very key specific epitope in the form of serum TK1.

The fourth peptide fragment includes the sequence SEQ ID NO: 4 shown as follows: NCPVPGKPGE (SEQ ID NO: 4).

The fifth peptide fragment includes the sequence SEQ ID NO: 5 shown as follows: PVPGKPGEAV (SEQ ID NO: 5).

The inventor used the ELISA method to detect the IgY-TK1 polyclonal antibody prepared by immunization with the aforementioned antigen of the first peptide fragment at the carbon terminal. The results show that this antibody has an immune response to the four specific epitopes and access epitopes (GQPAGPDNKEN195˜205) mentioned above. But antibodies from different hens showed different percentages of immune responses to the four specific epitopes and accessible epitopes. The epitope of the third peptide fragment at the carbon terminal showed the main and strongest immune response. Specifically, for each hen immunized with the C-terminal polypeptide of human thymidine kinase 1 as the antigen, the immune response of the antibody obtained is different. The first polyclonal antibody and the second polyclonal antibody can be obtained by immunizing two chickens of the same genus. Antibodies are polyclonal antibodies that recognize different epitope responses and realize the pairing of the sandwich method of this kit. According to an embodiment of the present disclosure, the epitopes recognized by the first polyclonal antibody and the second polyclonal antibody are selected from at least 3-4 of the above-mentioned first to fifth peptide fragments of thymidine kinase 1, and there must be the third peptide fragment at the carbon terminal.

According to an embodiment of the present disclosure, the kit further includes a substrate luminescence catalyst coupled with a marker identifier that specifically recognizes the marker; and a luminescence substrate that emits a light signal under action of the substrate luminescence catalyst. The marker is specifically identified by the marker identifier to form a specific signal amplification system, which improves the detection sensitivity and repeatability and shortens the detection time. According to a specific embodiment of the present disclosure, the marker is biotin, and the marker identifier is streptavidin. As a result, the use of the biotin-streptavidin high-specific signal amplification system significantly improves the detection sensitivity and repeatability, and the reaction time is shortened from 7 hours in traditional detection methods to 50 minutes.

According to an embodiment of the present disclosure, the luminescence substrate is APS-5.

According to an embodiment of the present disclosure, the substrate luminescence catalyst may be alkaline phosphatase, and the substrate luminescence catalyst coupled to the marker identifier may be alkaline phosphatase-labeled streptavidin. The substrate luminescence catalyst may also be horseradish peroxidase, and the substrate luminescence catalyst coupled to the marker identifier may be streptavidin labeled with avidin horseradish peroxidase.

According to an embodiment of the present disclosure, the solid carrier is magnetic particles. Thus, by coupling the polyclonal antibody to the magnetic particles, the immunochemiluminescence technology and the automatic magnetic particle immunoluminescence analyzer are combined, providing a nearly homogeneous reaction system. The use of a highly specific signal amplification system of luminescence marker-label identifier greatly improves the detection sensitivity and repeatability, and the reaction time is shortened from 7 hours in traditional detection methods to 50 minutes.

According to an embodiment of the present disclosure, the particle size of the magnetic particles is 2-5 microns. Further, the particle size of the magnetic particles is 3 microns. Therefore, the particle size of the magnetic particles is suitable, and the matching degree with the automatic chemiluminescence immunoassay is better. In the case of matching magnetic fields, a higher degree of separation can be obtained, and the magnetic particles in this size range have a high degree of binding to the antibody, which is beneficial to improve the detection accuracy.

According to an embodiment of the present disclosure, a method for preparing the first antibody coupled with magnetic particles is as follows:

1) adding fully mixed tosyl magnetic particle concentrate into a reaction flask, placing the reaction flask in a magnetic field for 15-20 minutes, removing a supernatant after all the tosyl magnetic particles are absorbed by the magnetic field, adding 2-20 times the volume of magnetic particle activation buffer to the reaction flask, shaking and washing for 10 minutes, then placing the reaction flask in the magnetic field for 15-20 minutes, and removing the supernatant; repeating cleaning tosyl magnetic particles 2 times; finally, diluting the tosyl magnetic particle solution to 1-20 mg/ml, mixing well and setting aside;

2) adding the first antibody to the tosyl magnetic particle solution prepared in step 1) according to a mass ratio of tosyl magnetic particle solution: the first antibody=1000:1 to perform a ligation reaction, adding 1/10 to ½ of the total volume of the magnetic particle catalytic buffer, and reacting at 37° C. for 18 hours in a mixed state;

3) adding 10% BSA of 1/50˜ 1/10 of the total volume of the solution to the magnetic particle solution prepared in step 2), and reacting at 37° C. for 6 hours in a mixed state;

4) placing the reaction flask in a magnetic field for 15 minutes, cleaning the tosyl magnetic particles 3 times with magnetic particle cleaning solution after the tosyl magnetic particles are absorbed into the magnetic field, then adjusting to 1 mg/ml and storing at 2-8° C. to prepare the first antibody coupled with magnetic particles.

The method for preparing the magnetic particle activation buffer is dissolving 5.18˜7.36 g of boric acid in 900 ml of deionized water, adjusting pH to 8-10 with NaOH, diluting to 1 L and filtering with 0.45 μm filter membrane. The method for preparing the magnetic particle catalytic buffer is dissolving 100-150 g of ammonium sulfate in 1 L of magnetic particle activation buffer, and filtering with 0.45 μm filter membrane after complete dissolution; the magnetic particle cleaning solution is TBST buffer with pH 7.4. The prepared first antibody coupled with the magnetic particle is also called the magnetic particle reagent coupled with the first antibody.

According to an embodiment of the present disclosure, the kit further includes a calibrator, a quality control product, an anti-reagent, a diluent and a washing solution.

The washing solution is TBST buffer with pH 7.4.

The method for preparing the diluent is dissolving 0.1 g-10 g blocking agent in 1 L Tris buffer, adding 0.1 ml -5 ml preservative, completely dissolving and filtering with 0.22 μm filter membrane.

The anti-reagent is prepared with anti-reagent buffer. The method for preparing the anti-reagent buffer is: dissolving 0.1 g-10 g blocking agent in 1 L Tris buffer, adding 0.1 ml -5 ml preservative, completely dissolving and filtering with 0.22 μm filter membrane to prepare the anti-reagent buffer. Then a biotin is coupled with the second antibody to obtain a second antibody labeled with a biotin, and the second antibody labeled with the biotin is diluted with a reagent buffer to a final concentration of 0.05 μg/ml to 0.5 μg/ml. The anti-reagent may also be referred to as the second antibody labeled with the biotin.

According to an embodiment of the present disclosure, both the calibrator and quality control product are pure TK1. The calibrator includes 5 concentrations to determine the standard curve to calculate the concentration. The quality control product includes 2 concentrations. The quality control product is used to confirm the effectiveness of the reagent and whether the result of the test quality control product is within the specified range.

In order to facilitate the use of the kit of the embodiment of the present disclosure, a general method for determining the content of thymidine kinase 1 in serum by using the kit is provided herein, including the following steps:

1) adding the reagents in the kit into the automatic chemiluminescence analyzer in sequence;

2) drawing 1-10 μl sample, 10-100 μl diluent, 10-100 μl magnetic particle reagent, and 10-100 μl anti-reagent into the reaction cup through the automatic chemiluminescence analyzer, and reacting for 10 minutes at 37° C., performing magnetic separation through the cleaning device, discarding the supernatant, and cleaning the complex precipitate with the washing solution for 1 to 6 times;

3) adding 10-100 μl of alkaline phosphatase reagent labeled with streptavidin to the reaction cup, reacting for 10 minutes at 37° C., performing magnetic separation through the cleaning device, discarding the supernatant, and cleaning the complex precipitate with washing solution for 1 to 6 times;

4) adding 10-500 μl of the luminescent substrate to the reaction cup containing the complex precipitate, reacting for 1 to 5 minutes, and entering the dark box to read the luminescence value. The content of thymidine kinase 1 in the sample is positively correlated with the luminescence value, and the content of thymidine kinase 1 is calculated from the luminescence standard curve.

The technical principle of the kit of the embodiment of the present disclosure is: the magnetic particles coupled with the first antibody and the second antibody labeled with biotin combine with thymidine kinase 1 in the sample, calibrator or quality control product form a sandwich complex. Subsequently, alkaline phosphatase-labeled streptavidin reagent is added, and streptavidin specifically binds to biotin and plays a signal amplification effect. Under the action of an external magnetic field, the complex formed by the immune reaction is separated from the unbound other substances, and after the complex is cleaned, an enzymatic chemiluminescence substrate is added. The substrate is catalytically cleaved under the action of enzymes to form unstable excited state intermediate. When the excited state intermediate returns to the ground state, a photon is emitted to form a luminescence reaction, and the photon intensity is read by the photomultiplier tube of an automatic chemiluminescence immunoassay and converted into a digital signal. In the detection range, the luminescence intensity is proportional to the content of thymidine kinase 1 in the sample, and the concentration of thymidine kinase 1 in the sample can be calculated using the modified four-parameter Logistic equation fitting.

According to the kit provided by the present disclosure, the specificity of the detection is to determine the level of TK1 in tumor cell TK1 positive strains and TK1 negative strains through comparative analysis to verify the specificity of the kit provided by the present disclosure for detecting TK1.

According to another aspect of the present disclosure, the present disclosure provides a use of the kit in the detection of thymidine kinase 1. Detection of thymidine kinase 1 in serum can reflect the abnormal proliferation of cells. Therefore, it can early warning of potential abnormal cell proliferation in the physical examination population earlier than the imaging test, warning the subject of the risk of malignant tumors, and it can also be used to detect the proliferation rate of small tumors.

According to another aspect of the present disclosure, the present disclosure provides a use of the kit for assessing risk of small malignant tumors and tumors/precancerous diseases that cannot be detected by images. That is to say, the kit can evaluate the effectiveness of early detection of malignant tumors and predicting the risky process of tumor development. In other words, in the early stage of the tumor, due to the small size of the tumor, it is difficult to detect by imaging. Through the kit of the embodiment of the present disclosure, through the detection and analysis of thymidine kinase 1, combined with other medical tests, the onset of tumor can be judged early, and can be used for tumor risk assessment. Specifically, the test method using the kit includes using the method or the kit according to the embodiment to determine the level of serum TK1 substance in the body sample from the subject of the physical examination population. The level of serum TK1 substance in the serum sample is then compared with the level of serum TK1 substance previously measured in the subject. If the measured serum TK1 level is elevated, it indicates that the risk of tumor-related disease progression in the subject has increased.

According to another aspect of the present disclosure, the present disclosure provides a method for determining abnormal cell proliferation in a subject. The method is especially suitable for people undergoing physical examination. According to an embodiment of the present disclosure, the method includes: using the kit described above to determine a content of thymidine kinase 1 in a serum of the subject; and evaluating whether the cell proliferation in the subject is abnormal based on the content of the thymidine kinase 1.

According to a specific embodiment of the present disclosure, the level of normal or tumor cell proliferation is measured and compared with the measured serum TK1 level to determine whether the subject has normal or baseline cell proliferation or elevated cell proliferation.

The additional aspects and advantages of the present disclosure will be partly given in the following description, and part will become obvious from the following description, or be understood through the practice of the present disclosure.

### BRIEF DESCRIPTION OF THE DRAWINGS

The above and/or additional aspects and advantages of the present disclosure will become obvious and easy to understand from the description of the embodiments in conjunction with the following drawings.

FIG. 1 shows a standard curve diagram according to an embodiment of the present disclosure.

FIG. 2 shows results of the detection of serum TK1 concentration distribution and percentage of population according to an embodiment of the present disclosure, A is kit 1, B is kit 2, and C is kit 3.

FIG. 3 shows a result diagram of correlation between dilution concentration of the lysate of tumor cell TK1 positive cell line and expression of TK1 in the kit according to the embodiment of the present disclosure.

FIG. 4 shows a schematic diagram of correlation between human STK1p concentration and tumor growth according to an embodiment of the present disclosure.

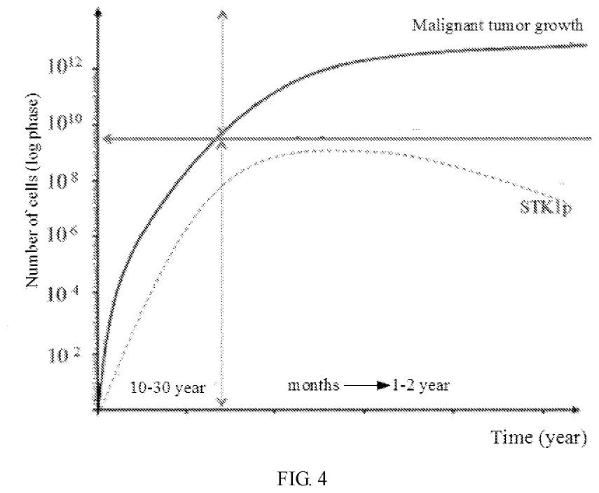

### DETAILED DESCRIPTION OF THE EMBODIMENTS

The embodiments of the present disclosure are described in detail below, and examples of the embodiments are shown in the accompanying drawings. The same or similar reference numerals indicate the same or similar elements or elements with the same or similar functions. The embodiments described below with reference to the drawings are exemplary, and are only used to explain the present disclosure, but should not be understood as limiting the present disclosure.

It should be noted that the terms “first” and “second” are only used for descriptive purposes, and cannot be understood as indicating or implying relative importance or implicitly indicating the number of indicated technical features. Thus, the features defined with “first” and “second” may explicitly or implicitly include one or more of these features. Further, in the description of the present disclosure, unless otherwise specified, “a plurality of” means two or more.

The present disclosure will be described below with reference to specific embodiments. It should be noted that these embodiments are merely illustrative and should not be understood as limiting the present disclosure.

The solution of the present disclosure will be explained below in conjunction with examples. Those skilled in the art will understand that the following embodiments are only used to illustrate the present disclosure and should not be regarded as limiting the scope of the present disclosure. Where specific techniques or conditions are not indicated in the embodiments, the procedures shall be carried out in accordance with the techniques or conditions described in the literature or in accordance with the product specification. The reagents or instruments used without the manufacturer's indication are all conventional products that can be purchased commercially, for example, purchased from Sigma.

### Embodiment 1

The kit of the embodiment of the present disclosure includes a calibrator, a quality control product, a blocking agent, a magnetic particle reagent coupled with a first antibody, and a second antibody labeled with a biotin, a streptavidin-labeled alkaline phosphatase, a luminescence substrate, an anti-reagent, a diluent, a washing solution. The preparation method is as follows.

1. Coupling Magnetic Particle Reagent with the First Antibody

1) Adding fully mixed tosyl magnetic particle concentrate into a reaction flask, placing the reaction flask in a magnetic field for 15 minutes, removing a supernatant after all the tosyl magnetic particles are absorbed by the magnetic field, adding 10 times the volume of magnetic particle activation buffer to the reaction flask, shaking and washing for 10 minutes, then placing the reaction flask in the magnetic field for 15 minutes, and removing the supernatant; repeating cleaning tosyl magnetic particles 2 times; finally, diluting the tosyl magnetic particle solution to 10 mg/ml, mixing well and setting aside;

2) Adding the first antibody to the tosyl magnetic particle solution prepared in step 1) according to a mass ratio of tosyl magnetic particle solution: the first antibody=100:1 to perform a ligation reaction, adding 1/10 of the total volume of the magnetic particle catalytic buffer, and reacting at 37° C. for 18 hours in a mixed state;

3) Adding 10% BSA of 1/20 of the total volume of the solution to the magnetic particle solution prepared in step 2), and reacting at 37° C. for 6 hours in a mixed state;

4) Placing the reaction flask in a magnetic field for 15 minutes, cleaning the tosyl magnetic particles 3 times with magnetic particle cleaning solution after the tosyl magnetic particles are absorbed into the magnetic field, then adjusting to 1 mg/ml and storing at 4° C. to prepare the first antibody coupled with magnetic particles.

The method for preparing the magnetic particle activation buffer is dissolving 5.18˜7.36 g of boric acid in 900 ml of deionized water, adjusting pH to 8-10 with NaOH, diluting to 1 L and filtering with 0.45 μm filter membrane.

The method for preparing the magnetic particle catalytic buffer is dissolving 100-150 g of ammonium sulfate in 1 L of magnetic particle activation buffer, and filtering with 0.45 μm filter membrane after complete dissolution.

The magnetic particle cleaning solution is TBST buffer with pH 7.4.

2. Second Antibody Labeled with the Biotin

using PBS to prepare a 2-5 mg second antibody solution, and use DMSO to prepare a 5-50 mM biotin solution, adding the biotin solution to the antibody solution and mixing well, reacting in an ice bath for 2 hours or at room temperature for 30 minutes to prepare the second antibody labeled with the biotin, and diluting the second antibody solution labeled with the biotin to 0.2 ug/ml. The second antibody is a polyclonal antibody obtained by immunizing the chicken with the sequence shown in SEQ ID NO: 6 as an antigen.

3. Luminescence Substrate

APS-5 (purchased from Wason Biotech)

4. Blocking Agent

Skimmed milk powder

5. Diluent

dissolving 0.1 g-10 g blocking agent in 1 L Tris buffer, adding 0.1 ml -5 ml preservative, completely dissolving and filtering with 0.22 μm filter membrane.

6. Anti-Reagent

diluting the biotinylated anti-thymidine kinase 1-IgY polyclonal antibody to a final concentration of 0.2 ug/ml.

7. Washing Solution

TBST buffer with PH 7.4 (30 times concentrated solution)

8. Streptavidin-Labeled Alkaline Phosphatase

Streptavidin-labeled alkaline phosphatase is prepared by purchasing Invitrogen streptavidin-labeled alkaline phosphatase and diluting 50,000 times with a diluent.

### Embodiment 2

To evaluate the kit for detecting serum TK1 obtained in embodiment 1, the specific method is as follows:

1. Sample preparation:

(1) Basic serum: 1 ml serum (concentration 2.2 pmol/L)+0.1 ml distilled water;

(2) Recovery sample 1: Serum 1 ml+0.1 ml antigen solution (concentration 11 pmol/L);

(3) Recovery sample 2: Serum 1 ml+0.1 ml antigen solution (concentration 80 pmol/L).

(4) Quality control sample 1: Take 1 ml standard product 1+5 ml standard product diluent (final concentration is 2.2 pmol/L).

(5) Quality control sample 2: Take 1 ml standard product 1+1 ml standard product diluent (final concentration is 10 pmol/L).

2. Preparation before the experiment

1) Taking a bottle of washing solution and dilute it 30 times with purified water;

2) Mixing the magnetic particle reagent thoroughly until there is no visible precipitation.

3. Experimental method, this kit is automatically completed by an automatic chemiluminescence analyzer, or can be completed manually.

1) Adding 10 μl sample to be tested, 60 μl sample diluent and 30 μl magnetic particle reagent to the detection tube, and incubating at 37° C. for 10 minutes;

2) Adding a magnetic field to settle the magnetic particles in the reaction system in the detection tube, removing the supernatant, and removing the magnetic field after washing for many times;

3) Adding 100 μl of anti-reagent to the washed system in step 2) and incubating at 37° C. for 10 minutes;

4) Adding a magnetic field to settle the magnetic particles in the reaction system in the detection tube, removing the supernatant, and removing the magnetic field after washing for many times;

5) Adding 100 μl of streptavidin-labeled alkaline phosphatase reagent to the washed system in step 4) and incubating at 37° C. for 10 minutes;

6) Adding a magnetic field to settle the magnetic particles in the reaction system in the detection tube, removing the supernatant, and removing the magnetic field after washing for many times;

7) Adding 200 μl of chemiluminescent substrate, mixing well, reacting for 2 minutes at room temperature and avoid light, and detecting the relative luminescence intensity (RLU).

4. Standard curve

1) Preparation of standards

Dissolving the 31 peptide antigen freeze-dried powder (purchased) in the standard diluent (10 mM Na₂HPO₄, 10 mM NaH₂PO₄, 150 mM NaCl, 1% BSA, 5% glycerol, pH 7.4) to prepare a concentration of 1 mg/ml, using Kit 3 to test and confirm the concentration of the antigen solution, adjusting the concentration to 20 pmol/L (standard 1) after the test. Continue to use the standard diluent to dilute standard 1 3 times to obtain standard 2, and dilute standard 2 with standard diluent 3 times to obtain standard 3, Dilute standard 3 twice to obtain standard 4, and use standard diluent as standard 5. The theoretical concentrations of the obtained standards 1-5 are: 20 pmol/L, 6.6 pmol/L, 2.2 pmol/L, 1.1 pmol/L and 0 pmol/L.

2) Drawing the standard curve

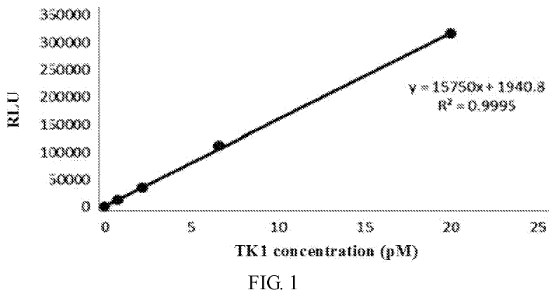

Using kit 1 to test 5 standard products to obtain the luminescence value, and drawing the standard curve according to the theoretical concentration combined with the luminescence value, as shown in FIG. 1, the R value is 0.999.

5. Recovery rate experiment

The basic serum, the recovery sample 1 and the recovery sample 2, each sample were repeatedly tested 3 times according to the method of the present disclosure, and the results are as follows:

| | Measurement | Recovery | Added | Recovery | Recovery | Acceptable |
|---|---|---|---|---|---|---|
| | value | mean value | concentration | concentration | rate | range |
| Basic | 2.239 | 2.245 | | | | |
| sample | 2.269 | | | | | |
| | 2.228 | | | | | |
| Recovery | 3.321 | 3.336 | 1.100 | 1.091 | 99.18% | 95-105% |
| sample 1 | 3.374 | | | | | |
| | 3.314 | | | | | |
| Recovery | 10.224 | 10.272 | 8.000 | 8.026 | 100.33% | 95-105% |
| sample 2 | 10.31 | | | | | |
| | 10.281 | | | | | |

6. Precision experiment

Quality control sample 1 (2.2 pmol/L) and quality control sample 2 (10 pmol/L) were repeated 20 times according to the method of the present disclosure. The results are as follows:

| | |
|---|---|
| quality control sample 1 | quality control sample 2 |
| (2.2 pmol/L) | (10 pmol/L) |

| measurment | | measurment | |
|---|---|---|---|
| value | CV | value | CV |
| 2.279 | 1.01% | 10.304 | 0.44% |
| 2.270 | | 10.363 | |
| 2.279 | | 10.290 | |
| 2.233 | | 10.199 | |
| 2.224 | | 10.208 | |
| 2.246 | | 10.192 | |
| 2.252 | | 10.199 | |
| 2.258 | | 10.182 | |
| 2.264 | | 10.214 | |
| 2.270 | | 10.209 | |
| 2.276 | | 10.219 | |
| 2.282 | | 10.214 | |
| 2.288 | | 10.205 | |
| 2.294 | | 10.216 | |
| 2.300 | | 10.193 | |
| 2.306 | | 10.187 | |
| 2.296 | | 10.227 | |
| 2.299 | | 10.194 | |
| 2.302 | | 10.215 | |
| 2.278 | | 10.219 | |

The experimental results show that the kit of the embodiment of the present disclosure has a higher recovery rate and precision.

### Embodiment 3

In this embodiment, three kits were used to detect TK1 in the sera of 148 cases of physical examination. These three kits used 3 different antibodies and methods respectively, as follows:

Kit 1: The kit is obtained in embodiment 1, and matches with an automatic chemiluminescence immunoassay analyzer for testing;

Kit 2: The kit contains the hTK1-IgY pAb obtained from the 31 peptide of human TK1 immunization of embodiment 1 and the sandwich IgY+IgG formed by the mouse anti-hTK IgG monoclonal antibody obtained from the 31 peptide of hTK1 immunization of embodiment 1, and matches the kit test of the automatic chemiluminescence immunoassay analyzer;

Kit 3: Dot blot enhanced chemiluminescence (ECL) immunoassay kit based on hTK1-IgY pAb (purchased from SINO-SWED TONGKANG BIO-TECH (SHENZHEN) LIMITED, trade name: thymidine kinase 1 (TK1) cell cycle analysis kit).

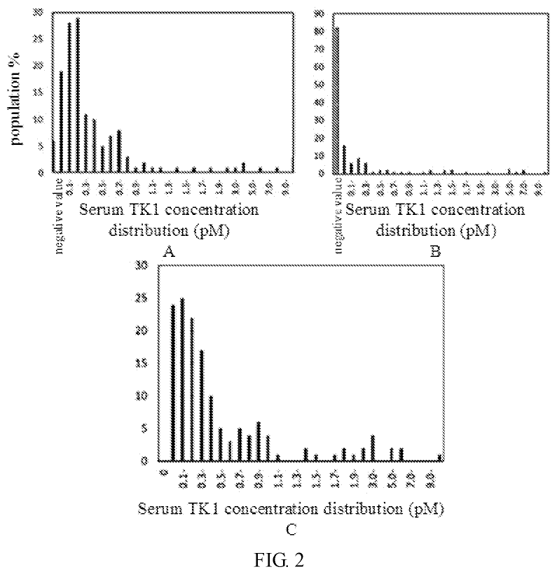

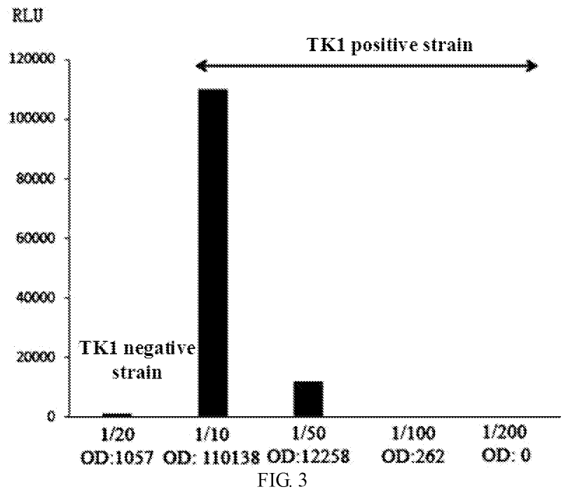

The test found that the coincidence rate of the test results of kit 1 and kit 3 was 73%, while the coincidence rate of the test results of kit 2 and kit 3 was only 54%. The specific experimental results are shown in FIG. 2. The distribution of serum TK1 concentration values from low to high values of individuals in the human population is analyzed by using Excel to make a graph. The characteristics are the same as those published in the current routine physical examination screening population survey research. FIG. 3 shows that the distribution characteristics of kit 1 and kit 3 are approximately normal distribution, with the main peak between 0.2-0.3 pmol/L. When the concentration of STK1p gradually increased from 0.6 pmol/L to 2 pmol/L, a small continuous decrease tail peak was found. The observation that the serum TK1 concentration distribution of the physical examination population presents an approximate normal distribution is an important finding. It is pointed out that the distribution of serum TK1p concentration conforms to the natural law distribution, and its determination sensitivity can reach 0.1-0.2 pmol/L. But it is observed that the distribution of serum TK1 concentration was between 0.6 pmol/L-2.0 pmol/L−>2.0 pmol/L, there is a continuous small tail peak that rises. The continuous small tail peaks of this increase reflect that individuals in this segment may have an increased risk of developing precancerous diseases/malignant tumors. However, in kit 2, the serum TK1 value is 83% negative, and the serum TK1 value does not show a main peak between 0.2-0.3 pmol/L, which is an approximate normal distribution characteristic, indicating that the sensitivity is not high.

### Embodiment 4

In this embodiment, Kit 1 and Kit 2 in embodiment 3 are used to perform TK1 test on the cell lysates of TK1 positive cell lines and negative cell lines, as follows:

The TK1 positive cell line (human colon tumor TK1⁺: ht29) and TK1 negative cell line (human colon tumor: 143bTK−, TK1 gene knockout cells) were cultured to the logarithmic growth phase, and the concentration is 1*10⁷/ml. After centrifuging 1 ml of cell suspension, e the supernatant was removed and 1 ml of cell lysate (50 mM Tris, 150 mM NaCl, 1 mM EDTA, 1% NP40) was added, and treated at 4° C. for 20 min, centrifuged at 15,000 rpm for 10 min, and the supernatant was taken. The negative cell line lysate and positive cell line lysate were diluted with PBS respectively, and after the negative cell line lysate was diluted 10 times, the positive cell line lysate was diluted 10 times, 50 times, and 100 times, respectively, the cell lysate was tested using kit 1 and kit 2 respectively.

The test results of kit 1 are shown in FIG. 3. The dilution concentration of TK1 positive cell line lysate is related to the expression of TK1. However, the results of kit 2 were abnormal, and there was no correlation between the dilution concentration of TK1 positive cell line lysate and the expression of TK1 (not shown). According to the data of the TK1 value of the positive cell line given in the figure, it is calculated that a tumor cell growing in the logarithmic phase contains approximately 0.021 pg of TK1 protein. If the total protein of a tumor cell is 200 pg, it is only 0.01% (0.021 pg/200 pg total protein=0.01%). Therefore, the content of TK1 in a growing tumor cell is very low, and only a highly sensitive detection system can perform accurate detection. When the TK1 in the tumor cells is released into the blood, it is calculated according to the volume of about 5000 ml of human blood, 52 million tumor-growing cells are needed to detect the TK1 value in the serum. However, the existing imaging system requires 1 billion tumor cells to reach a small tumor with a diameter of about 1 mm before it can be detected by imaging.

The kit 1 detection system of the present disclosure has high sensitivity. In the case of micro malignant tumors that are not detectable on images, an increase in the serum TK1 value is detected, warning the patient to have precancerous diseases with abnormal proliferating cells/micro malignant tumors that are not detectable on images. According to FIG. 2 and FIG. 3, the correlation diagram 4 between the value of STK1p and the number of tumor cells (proliferation rate) and time is drawn. Below the image detection threshold line indicates precancerous basic or small malignant tumors that are not detectable or untouchable in the image, and above the image detection threshold line indicates malignant tumors that are detectable or touchable in the image. The kit of the embodiment of the present disclosure can detect precancerous diseases of small malignant tumors/abnormally proliferating cells with STK1>2 pmol/L and undetectable images. As shown in the Figure, serum TK1 expression is closely related to the number of tumor cells in the early and middle phases of tumor growth (less than 1 billion tumor cells). However, in many cases, serum TK1 expression decreases in the late phase of tumor growth. This is because although the tumor tissue increases and reaches a larger volume in the later phase of tumor growth, it can be detected by imaging. However, different degrees of tissue necrosis will occur in the center of such large tumor tissues, which leads to a decrease in the proliferation rate and a correspondingly low concentration of STK1p. Furthermore, the kits of the embodiments of the present disclosure can be used for early detection of small latent malignant tumors that are not detectable on images by detecting the elevated value of serum TK1. At the same time, the risk assessment of the precancerous disease process of abnormally proliferating cells can also be used.

In the description of this Specification, description with reference to the terms “an embodiment”, “some embodiments”, “examples”, “specific examples”, or “some examples” etc. mean that the specific features, structures, materials or characteristics described in combination with the embodiment or example are included in at least one embodiment or example of the present disclosure. In this Specification, the schematic representations of the above terms do not necessarily refer to the same embodiment or example. Moreover, the described specific features, structures, materials or characteristics may be combined in any one or more embodiments or examples in a suitable manner.

Although the embodiments of the present disclosure have been shown and described, those of ordinary skill in the art can understand various changes, modifications, substitutions and modifications can be made to these embodiments without departing from the principle and purpose of the present disclosure. The scope of the present disclosure is defined by the claims and their equivalents.

What is claimed is:

1. A kit, comprising:
a first polyclonal antibody that has been immobilized or is suitable for immobilization on a solid carrier; and
a second polyclonal antibody labeled with a marker,
wherein both the first polyclonal antibody and the second polyclonal antibody are chicken anti human thymidine kinase 1 IgY-polyclonal antibodies, and both the first polyclonal antibody and the second polyclonal antibody are suitable for specifically binding to thymidine kinase 1.

2. The kit of claim 1, wherein epitopes recognized by the first polyclonal antibody and the second polyclonal antibody include:
a third peptide fragment at a carbon terminal, the third peptide fragment including a sequence shown in SEQ ID NO: 3; and
at least two peptide fragments selected from the following peptide fragments:
a first peptide fragment at the carbon terminal, the first peptide fragment including a sequence shown in SEQ ID NO: 1;
a second peptide fragment at the carbon terminal, the second peptide fragment including a sequence shown in SEQ ID NO: 2;
a fourth peptide fragment at the carbon terminal, the fourth peptide fragment including a sequence shown in SEQ ID NO: 4;
a fifth peptide fragment at the carbon terminal, the fifth peptide fragment including a sequence shown in SEQ ID NO: 5.

3. The kit of claim 1, wherein the first polyclonal antibody and the second polyclonal antibody are obtained by immunizing different chickens with an antigen, and the antigen is a polypeptide at a carbon terminal of human thymidine kinase 1.

4. The kit of claim 3, wherein the antigen includes a sequence shown in SEQ ID NO: 6.

5. The kit of claim 1, further comprising:
a substrate luminescence catalyst coupled with a marker identifier that specifically recognizes the marker; and
a luminescence substrate that emits a light signal under action of the substrate luminescence catalyst.

6. The kit of claim 5, wherein the marker is biotin, and the marker identifier is streptavidin.

7. The kit of claim 1, wherein the solid carrier is magnetic particles.

8. The kit of claim 1, further comprising a calibrator, a quality control product, an anti-reagent, a diluent and a washing solution.

9. A use of the kit of claim 1 for detecting thymidine kinase 1.

10. A use of the kit of claim 1 for assessing risk of small malignant tumors and tumors/precancerous diseases that cannot be detected by images.

11. A method for determining abnormal cell proliferation in a subject, comprising:
using the kit of claim 1 to determine a content of thymidine kinase 1 in a serum of the subject; and
evaluating whether the cell proliferation in the subject is abnormal based on the content of the thymidine kinase 1.
